SEQUENCE LISTING (69 sequences)

SEQ ID NO: 1 - DNA, 1923 bp, Magnaporthe oryzae
  source 1..1923: mol_type = genomic DNA
atgcatctgatccagagcctcgtgtgggctctcccattggcactgccactcgtggctgccgatattgaggatgtcggtgacaagccccgcaagttcgaatacaccatcacttgggaaaaggggtccccggatggtttcgagcgtgagatggccttcatcaacggaaggtttcctgggccggtcgttgaggcaaaccagggagacaacatagagattgtcgtcaccaaccagatgcctttcaacacaaccatccactggcatggtattgagcaaaaaggtaccccgtggtcagatggcgtaccgggcctcacgcagcgctacatcatgcccggcaagaatttcacctacaagtggaaggcggaacagtacggcagctattggtaccacgcccacgcacgcggccagattgacgatggcttgtacggcggtatcctcatccacccgagcaacaccacgcaagactcgctcagcctgatcgcccggaacgcctcccagctcgctgcgatgcggaccgccgtgcgcaacgttcagcccatcatcctctccgactggcgacacctcaagtccatggatgcctggcagataaacgtcgactccaagctcgagacgccttgctacgactcccttctcgtcaacggcaagggccaggtcaactgctggtctcagcaaaagattaaccagcttacgaacggcatccaaagacgattcctgcagactgccaacatgcagcagatgaccgccaagggatgtctcccagccaaggctgtgcaaatgacctttgcccggaacatgcccggaaacctctcggcgattcccaaggaggtctttgatgtttgcacaccaacccagggtcagcaaagtgttatcaaagtcaaacaggctgcaggcgcaaccgagtactggcaagcctttgacattgtcggcacgtttggtctcgtcaacgctgctgtctcgatggacgagcaccccatgtacgtttatgctgtcgacggtggctacatccagccgcagttggtccaggccatctccgtcagcaacgccgaccgctactcggtcctggtcaaggtcgacaggcccggcgactacaccctccgcgtcgcctccaactccgtcatccagatgatgagcgtgactgccactgtccgcatcctaggagccaactccgccgagacggccgacggctcagacaatttcatcacccccacgagcgccaacgccactagcgccagcggcagctcccccgccccctccaacgtccgccgccagctgcccggcgccagctcgacgccctacatcaacgacgtcggacagggcacaagccagagcgtcacctttttcagccagaacaaccagcgccagtacccggccgaccccccgggacagacggtcgcccagaccttcaagatgcagctccgccacgacggcagcgcctacaagtgggcgctcaacgacaccatctaccccctcaacgccgacgatgcctacccggtgctgttcaagcccccggtcaaccgccaggacggcaccaccatcacgacgaggaacaacacctgggtcgacttcatcttcgagtcgtccaccttcccgactcccccgcaccccgtccacaagcacggcacgcacatgtacctcatcggctccggccagggcaacttcacctggaacacggtcgccgaggccgcgcgtgccaacccgggccgcttcaacctgcagaacccgcccaagcgcgactcgttccagagcgtgcccgccgtcgtgcagcccagctggatcgccgtccgctacttctcctccgacccgggcccctggctgatgcactgccatatccagacccatctcgagggtggcatggccatggcgatcatcgagggccctgaggtatggccggcgacgccggagacttacctgaactacgatcaatag

SEQ ID NO: 2 - RNA, 21 nt, Synthetic construct
  source 1..21: mol_type = other RNA
gccatgatatagacgttgttt

SEQ ID NO: 3 - RNA, 21 nt, Synthetic construct
  source 1..21: mol_type = other RNA
acaacgtctatatcatggctt

SEQ ID NO: 4 - RNA, 21 nt, Synthetic construct
  source 1..21: mol_type = other RNA
gcgagttcatctacaaggttt

SEQ ID NO: 5 - RNA, 21 nt, Synthetic construct
  source 1..21: mol_type = other RNA
accttgtagatgaactcgctt

SEQ ID NO: 6 - RNA, 25 nt, Synthetic construct
  source 1..25: mol_type = other RNA
cagaagattgttgtctgcttggttt

SEQ ID NO: 7 - RNA, 25 nt, Synthetic construct
  source 1..25: mol_type = other RNA
aaaccaagcagacaacaatcttctg

SEQ ID NO: 8 - RNA, 21 nt, Synthetic construct
  source 1..21: mol_type = other RNA
cgacagtaatgaagctctatt

SEQ ID NO: 9 - RNA, 21 nt, Synthetic construct
  source 1..21: mol_type = other RNA
tagagcttcattactgtcgtt

SEQ ID NO: 10 - RNA, 25 nt, Synthetic construct
  source 1..25: mol_type = other RNA
ccaggacaatgattggtgtattcaa

SEQ ID NO: 11 - RNA, 25 nt, Synthetic construct
  source 1..25: mol_type = other RNA
ttgaatacaccaatcattgtcctgg

SEQ ID NO: 12 - RNA, 25 nt, Synthetic construct
  source 1..25: mol_type = other RNA
gccgcattgtctgggttcaagttta

SEQ ID NO: 13 - RNA, 25 nt, Synthetic construct
  source 1..25: mol_type = other RNA
taaacttgaacccagacaatgcggc

SEQ ID NO: 14 - RNA, 21 nt, Synthetic construct
  source 1..21: mol_type = other RNA
gccgtgtcgagagtataattt

SEQ ID NO: 15 - RNA, 21 nt, Synthetic construct
  source 1..21: mol_type = other RNA
attatactctcgacacggctt

SEQ ID NO: 16 - RNA, 21 nt, Synthetic construct
  source 1..21: mol_type = other RNA
ggctcagacaatttcatcatt

SEQ ID NO: 17 - RNA, 21 nt, Synthetic construct
  source 1..21: mol_type = other RNA
tgatgaaattgtctgagcctt

SEQ ID NO: 18 - RNA, 21 nt, Synthetic construct
  source 1..21: mol_type = other RNA
gccgtgtcgagagtataattt

SEQ ID NO: 19 - RNA, 21 nt, Synthetic construct
  source 1..21: mol_type = other RNA
attatactctcgacacggctt

SEQ ID NO: 20 - RNA, 21 nt, Synthetic construct
  source 1..21: mol_type = other RNA
gtggtatcaagaccgatattt

SEQ ID NO: 21 - RNA, 21 nt, Synthetic construct
  source 1..21: mol_type = other RNA
atatcggtcttgataccactt

SEQ ID NO: 22 - RNA, 21 nt, Synthetic construct
  source 1..21: mol_type = other RNA
ctcgactacagcatcatcatt

SEQ ID NO: 23 - RNA, 21 nt, Synthetic construct
  source 1..21: mol_type = other RNA
tgatgatgctgtagtcgagtt

SEQ ID NO: 24 - RNA, 21 nt, Synthetic construct
  source 1..21: mol_type = other RNA
cgccctttgtctacaacaatt

SEQ ID NO: 25 - RNA, 21 nt, Synthetic construct
  source 1..21: mol_type = other RNA
ttgttgtagacaaagggcgtt

SEQ ID NO: 26 - RNA, 21 nt, Synthetic construct
  source 1..21: mol_type = other RNA
gaggtctcaagttcctcattt

SEQ ID NO: 27 - RNA, 21 nt, Synthetic construct
  source 1..21: mol_type = other RNA
atgaggaacttgagacctctt

SEQ ID NO: 28 - RNA, 21 nt, Synthetic construct
  source 1..21: mol_type = other RNA
cgatcgcaaagacaaccaatt

SEQ ID NO: 29 - RNA, 21 nt, Synthetic construct
  source 1..21: mol_type = other RNA
ttggttgtctttgcgatcgtt

SEQ ID NO: 30 - RNA, 21 nt, Synthetic construct
  source 1..21: mol_type = other RNA
gaaccaccgaaaggataattt

SEQ ID NO: 31 - RNA, 21 nt, Synthetic construct
  source 1..21: mol_type = other RNA
attatcctttcggtggttctt

SEQ ID NO: 32 - RNA, 21 nt, Synthetic construct
  source 1..21: mol_type = other RNA
gagcgacggtttgagaaattt

SEQ ID NO: 33 - RNA, 21 nt, Synthetic construct
  source 1..21: mol_type = other RNA
atttctcaaaccgtcgctctt

SEQ ID NO: 34 - RNA, 21 nt, Synthetic construct
  source 1..21: mol_type = other RNA
ccgtgaaccaatactttattt

SEQ ID NO: 35 - RNA, 21 nt, Synthetic construct
  source 1..21: mol_type = other RNA
ataaagtattggttcacggtt

SEQ ID NO: 36 - RNA, 21 nt, Synthetic construct
  source 1..21: mol_type = other RNA
gggcggtatagtatgtctatt

SEQ ID NO: 37 - RNA, 21 nt, Synthetic construct
  source 1..21: mol_type = other RNA
tagacatactataccgccctt

SEQ ID NO: 38 - RNA, 21 nt, Synthetic construct
  source 1..21: mol_type = other RNA
gcacggcacgcacatgtacct

SEQ ID NO: 39 - RNA, 21 nt, Synthetic construct
  source 1..21: mol_type = other RNA
gtacatgtgcgtgccgtgctt

SEQ ID NO: 40 - RNA, 21 nt, synthetic construct
  source 1..21: mol_type = other RNA
ggaggtctttgatgtttgcac

SEQ ID NO: 41 - RNA, 21 nt, synthetic construct
  source 1..21: mol_type = other RNA
gcaaacatcaaagacctcctt

SEQ ID NO: 42 - RNA, 21 nt, synthetic construct
  source 1..21: mol_type = other RNA
gttcgaatacaccatcacttg

SEQ ID NO: 43 - RNA, 21 nt, synthetic construct
  source 1..21: mol_type = other RNA
agtgatggtgtattcgaactt

SEQ ID NO: 44 - RNA, 21 nt, synthetic construct
  source 1..21: mol_type = other RNA
gctcgagacgccttgctacga

SEQ ID NO: 45 - RNA, 21 nt, synthetic construct
  source 1..21: mol_type = other RNA
gtagcaaggcgtctcgagctt

SEQ ID NO: 46 - RNA, 21 nt, synthetic construct
  source 1..21: mol_type = other RNA
gtccatggatgcctggcagat

SEQ ID NO: 47 - RNA, 21 nt, synthetic construct
  source 1..21: mol_type = other RNA
ctgccaggcatccatggactt

SEQ ID NO: 48 - RNA, 21 nt, synthetic construct
  source 1..21: mol_type = other RNA
gctcaacgacaccatctactt

SEQ ID NO: 49 - RNA, 21 nt, synthetic construct
  source 1..21: mol_type = other RNA
gtagatggtgtcgttgagctt

SEQ ID NO: 50 - RNA, 21 nt, synthetic construct
  source 1..21: mol_type = other RNA
ggcaagcctttgacattgttt

SEQ ID NO: 51 - RNA, 21 nt, synthetic construct
  source 1..21: mol_type = other RNA
acaatgtcaaaggcttgcctt

SEQ ID NO: 52 - RNA, 21 nt, synthetic construct
  source 1..21: mol_type = other RNA
gacttcatcttcgagtcgttt

SEQ ID NO: 53 - RNA, 21 nt, synthetic construct
  source 1..21: mol_type = other RNA
acgactcgaagatgaagtctt

SEQ ID NO: 54 - RNA, 21 nt, synthetic construct
  source 1..21: mol_type = other RNA
cttacctgaactacgatcatt

SEQ ID NO: 55 - RNA, 21 nt, synthetic construct
  source 1..21: mol_type = other RNA
tgatcgtagttcaggtaagtt

SEQ ID NO: 56 - RNA, 21 nt, synthetic construct
  source 1..21: mol_type = other RNA
agcaaagtgttatcaaagtca

SEQ ID NO: 57 - RNA, 21 nt, synthetic construct
  source 1..21: mol_type = other RNA
tgactttgataacactttgct

SEQ ID NO: 58 - RNA, 21 nt, synthetic construct
  source 1..21: mol_type = other RNA
gcaagaatttcacctacaatt

SEQ ID NO: 59 - RNA, 21 nt, synthetic construct
  source 1..21: mol_type = other RNA
ttgtaggtgaaattcttgctt

SEQ ID NO: 60 - RNA, 21 nt, synthetic construct
  source 1..21: mol_type = other RNA
ccaaggaggtctttgatgttt

SEQ ID NO: 61 - RNA, 21 nt, synthetic construct
  source 1..21: mol_type = other RNA
acatcaaagacctccttggtt

SEQ ID NO: 62 - RNA, 21 nt, synthetic construct
  source 1..21: mol_type = other RNA
ctgccgatattgaggatgttt

SEQ ID NO: 63 - RNA, 21 nt, synthetic construct
  source 1..21: mol_type = other RNA
acatcctcaatatcggcagtt

SEQ ID NO: 64 - RNA, 21 nt, synthetic construct
  source 1..21: mol_type = other RNA
ggctcagacaatttcatcatt

SEQ ID NO: 65 - RNA, 21 nt, synthetic construct
  source 1..21: mol_type = other RNA
tgatgaaattgtctgagcctt

SEQ ID NO: 66 - RNA, 21 nt, synthetic construct
  source 1..21: mol_type = other RNA
atggtattgagcaaaaaggtt

SEQ ID NO: 67 - RNA, 21 nt, synthetic construct
  source 1..21: mol_type = other RNA
cctttttgctcaataccattt

SEQ ID NO: 68 - RNA, 21 nt, synthetic construct
  source 1..21: mol_type = other RNA
gagacaacatagagattgttt

SEQ ID NO: 69 - RNA, 21 nt, synthetic construct
  source 1..21: mol_type = other RNA
acaatctctatgttgtctctt

(19) United States
(12) Patent Application Publication
(10) Pub. No.: US 2026/0041102 A1
(43) Pub. Date: Feb. 12, 2026

(54) SIRNA TARGETING KEY GENE OF MAGNAPORTHE ORYZAE FOR CONTROLLING RICE BLAST, AND USE THEREOF

(71) Applicant: Zhejiang Academy of Agricultural Sciences (ZAAS), Hangzhou (CN)

(72) Inventors: Jiaoyu WANG, Hangzhou (CN); Jing WANG, Hangzhou (CN); Ziqi LU, Hangzhou (CN); Zhongna HAO, Hangzhou (CN); Yingying CAI, Hangzhou (CN); Zhen ZHANG, Hangzhou (CN); Haiping QIU, Hangzhou (CN); Yanli WANG, Hangzhou (CN)

(21) Appl. No.: 19/292,244

(22) Filed: Aug. 6, 2025

(30) Foreign Application Priority Data
Aug. 9, 2024 (CN) 202411087506.0

(51) Int. Cl. A01N 63/60; A01N 25/02; A01N 25/28; A01P 3/00; C12N 15/113

(52) U.S. Cl. CPC A01N 63/60; A01N 25/02; A01N 25/28; A01P 3/00; C12N 15/113; C12N 2310/14

(57) ABSTRACT

A siRNA targeting a key gene of Magnaporthe oryzae for controlling rice blast and use thereof, and relates to the field of biotechnology. The siRNA targets silence of a MOHS1 gene (MGG_08523) of Magnaporthe oryzae. The siRNA molecule can delay and inhibit the formation of an appressorium of the Magnaporthe oryzae, thereby reducing the pathogenicity of the Magnaporthe oryzae, thereby providing a novel method for controlling the rice blast. The siRNA molecule targeting the MOHS1 gene is prepared into a nucleic acid pesticide, and the nucleic acid pesticide is sprayed onto leaves of Oryza sativa L., which effectively reduces the number and area of lesions of the rice blast on the leaves, in turn can be used for controlling the rice blast, and can avoid a tedious process of cultivating genetically modified crops and the concerns of consumer groups.

### CROSS-REFERENCE TO RELATED APPLICATIONS

The present application claims priority of claims priority of Chinese prior application No. 202411087506.0 filed on Aug. 9, 2024; all the content of which is incorporated by reference as a part of the present disclosure.

### REFERENCE TO AN ELECTRONIC SEQUENCE LISTING

The contents of the electronic sequence listing (Sequence Listing.xml; Size: 65,536 bytes; and Date of Creation: Aug. 6, 2025) is herein incorporated by reference in its entirety.

### BACKGROUND OF THE INVENTION

### Field of the Invention

The present disclosure relates to the field of biotechnology, and mainly relates to a siRNA targeting a key gene of Magnaporthe oryzae for controlling rice blast, and use thereof.

### Description of the Related Art

Oryza sativa L. is the staple food for more than half of the population in the world, which provides more than 20% of the calories consumed by humans and is critical to global food security. Rice blast, bacterial leaf blight and sheath blight are known as the three major diseases of Oryza sativa L. Among them, the rice blast is caused by Magnaporthe oryzae. The Magnaporthe oryzae can infect multiple sites of Oryza sativa L. According to the different times or tissues of disease occurrence, the rice blast can be divided into seedling blast, node blast, leaf blast and ear neck blast, etc., among which the ear neck blast is the main cause of yield loss in the Oryza sativa L. In addition to Oryza sativa L., the M. oryzae can also infect more than 50 species of Poaceae crops, including economically and agriculturally important crops such as Triticum spp., Hordeum vulgare, Zea mays and Poaceae weeds, etc.

The infection of Magnaporthe oryzae begins with conidia. When scattered onto the surfaces of leaves of plants such as Oryza sativa L., the conidia germinate and grow germ tubes under suitable temperature and humidity conditions. The top ends of the germ tubes swell and form a special infected structure, namely a hemispherical appressorium. As the appressorium forms and matures, a melanin layer is formed between a cell membrane and a cell wall, and a large amount of osmotic substances (e.g. glycerol) accumulate inside the appressorium, causing the turgor pressure inside the appressorium to gradually increase. Under the combined action of the melanin layer and the high turgor pressure, the high turgor pressure is converted into mechanical pressure to penetrate the surface of a host, forming an infection peg at the base of the appressorium, which penetrates the cuticle of the host to form primary infection hyphae. The rice blast fungus requires the melanin layer in the appressorium to produce and maintain the high turgor pressure required for plant penetration, where melanin synthesis requires the participation of multiple enzymes.

In recent years, RNAi pesticides (nucleic acid pesticides) developed based on RNAis have become a hot topic of research at home and abroad. dsRNAs or siRNAs are sprayed onto the surfaces of plants, and then directly or indirectly uptaken by target biological cells, causing in vivo RNAi responses, and in turn achieving the purpose of silencing target genes. This technology is also known as Spraying Induced Gene Silence (SIGS). The SIGS technology avoids the complex process and potential ecological risks of crop genetic modification, showing even greater development potential.

So far, although there have been many attempts to utilize the SIGS technology to control pathogenic fungi, there are very few nucleic acid molecules developed based on the key pathogenic genes of Magnaporthe oryzae, and no products have been reported, which in turn limits the application of the SIGS technology in the control of Magnaporthe oryzae.

### BRIEF SUMMARY OF THE INVENTION

In view of the aforementioned problems, the present disclosure designs a siRNA with a gene MGG_08523 (MOHS1) related to melanin synthesis of Magnaporthe oryzae as a target, analyzes the antibacterial effect of the obtained siRNA, and then applies the obtained siRNA onto leaves of Oryza sativa L. in the form of spray to inhibit the pathogenicity of the Magnaporthe oryzae, thereby achieving the purpose of controlling rice blast.

To achieve the aforementioned objective, the present disclosure adopts the following solutions: in an aspect, the present disclosure provides a nucleic acid pesticide for controlling rice blast, which is characterized in that, the nucleic acid pesticide includes a siRNA targeting silence of a MOHS1 gene of Magnaporthe oryzae, and the MOHS1 gene includes a nucleotide sequence as shown in SEQ ID NO: 1 in Sequence Listing.

In the present disclosure, firstly target genes for controlling Magnaporthe oryzae are screened, which are all involved in a process of regulating the growth and development of the Magnaporthe oryzae, and it has been found that when the siRNA (siMohs-10) is utilized to target the silence of the MOHS1 gene of the Magnaporthe oryzae, the formation proportion of an appressorium of the Magnaporthe oryzae can be significantly reduced. The MOHS1 gene is a gene related to melanin synthesis in the Magnaporthe oryzae.

In order to further improve the control efficiency of the Magnaporthe oryzae, the present disclosure has designed 17 pairs of siRNAs for the MOHS1 gene, and randomly selected 5 pairs of siRNAs to verify their inhibitory effects on the Magnaporthe oryzae, and has found that the siMohs-10 has the best inhibitory effect.

Specifically, after the siMohs molecule (i.e., the siRNA targeting the silence of the MOHS1 gene) is incubated with the Magnaporthe oryzae for 4 h, compared with siMohs-6 (SEQ ID NOs: 48-49), siMohs-12 (SEQ ID NOs: 58-59) and siMohs-17 (SEQ ID NOs: 68-69), the appressorium of the Magnaporthe oryzae applied with siMohs-7 (SEQ ID NOs: 50-51) and siMohs-10 (SEQ ID NOs: 16-17) has inhibited formation and smaller morphology; and further, the inhibitory effect of the siMohs-10 is better than that of the siMohs-7. In conclusion, the siMohs-10 molecule has the most significant inhibitory effect on the Magnaporthe oryzae.

In some embodiments, the siRNA includes a nucleotide sequence as shown in any one or more of SEQ ID NOs: 38-39 (siMohs-1), SEQ ID NOs: 40-41 (siMohs-2), SEQ ID NOs: 42-43 (siMohs-3), SEQ ID NOs: 44-45 (siMohs-4), SEQ ID NOs: 46-47 (siMohs-5), SEQ ID NOs: 48-49 (siMohs-6), SEQ ID NOs: 50-51 (siMohs-7), SEQ ID NOs: 52-53 (siMohs-8), SEQ ID NOs: 54-55 (siMohs-9), SEQ ID NOs: 16-17 (siMohs-10), SEQ ID NOs: 56-57 (siMohs-11), SEQ ID NOs: 58-59 (siMohs-12), SEQ ID NOs: 60-61 (siMohs-13), SEQ ID NOs: 62-63 (siMohs-14), SEQ ID NOs: 64-65 (siMohs-15), SEQ ID NOs: 66-67 (siMohs-16), and SEQ ID NOs: 68-69 (siMohs-17) in the in Sequence Listing.

In some embodiments, the siRNA includes a nucleotide sequence as shown in any one or more of SEQ ID NOs: 48-49, SEQ ID NOs: 50-51, SEQ ID NOs: 16-17, SEQ ID NOs: 58-59, and SEQ ID NOs: 68-69 in the Sequence Listing.

In some embodiments, the siRNA includes a nucleotide sequence as shown in any one or more of SEQ ID NOs: 16-17 in the Sequence Listing.

In some embodiments, the siRNA has a working concentration range of 1-100 μM.

In some embodiments, the siRNA is encapsulated by a nanomaterial.

The nanomaterial refers to a material with a size between 1-100 nanometers (nm) in at least one dimension, or a material with nanoscale structural characteristics. Its unique physical, chemical and biological properties originate from characteristics such as quantum effects, surface effects and a high specific surface area at the nanoscale. The nanomaterial may be naturally occurring (e.g. volcanic ash and biological molecules), but more often refers to an artificially designed and synthesized functional material that is widely applied in energy, electronics, medicine, environment and the like fields.

In some embodiments, the nanomaterial is a liposome.

The liposome is a an artificially synthesized nano- or micron-scale vesicle composed of one or more phospholipid bimolecular layers with an aqueous core encapsulated therein. It has a structure that mimics a natural lipid bilayer of a biofilm, can encapsulate hydrophilic or hydrophobic substances, and is widely applied in drug delivery, gene therapy, vaccine development, and cosmetics, etc.

In some embodiments, the liposome includes ionizable lipids, helper lipids, structural lipids, and polymer-modified lipids.

The ionizable lipids in the liposome are a class of cationic lipids with pH-responsive characteristics. Their charge states change with the pH of the environment. For example, they are crucial in gene therapy and mRNA vaccines (e.g. COVID-19 vaccines), can bind with nucleic acids (e.g. mRNAs) through electrostatic interactions and carry positive charges in the acidic endosomal environment, promoting the release of nucleic acids into the cytoplasm. The helper lipids impart the liposome with special functions, e.g. targeting, stabilizing or drug loading capacities. The structural lipids are used for adjusting the fluidity and stability of the lipid bilayer. The polymer-modified lipids are a class of functional lipids that covalently link polymer chains onto traditional lipid molecules. Their unique physicochemical properties significantly improve the performance of the liposome, for example extending the circulation time of the liposome, enhancing the stability of the liposome, improving the efficiency of targeted delivery of nucleic acids, controlling the release of contents, and improving biocompatibility.

On this basis, the present disclosure uses the liposome to encapsulate the siRNA (siMohs-10) that targets the silence of the MOHS1 gene. The liposome is a nucleic acid encapsulation reagent suitable for encapsulating an RNA in a variety of microfluidic encapsulation devices. By adjusting the type of the core chip, the flow rate, and the N/P ratio, lipid nanoparticles with homogeneous particle sizes and near-neutral surface potentials can be obtained. It has been found that after assembly with the nanomaterial, not only the inhibitory effect of the siMohs-10 on the Magnaporthe oryzae was prolonged, but also the formation rate of the appressorium of the Magnaporthe oryzae was greatly reduced, and the morphology of the appressorium becomes more deformed. This may be because the liposome can protect the siRNA from degradation to a certain extent and facilitate the siRNA to enter the interior of the Magnaporthe oryzae.

In some embodiments, the ionizable lipids include any one or more of DLin-MC3-DMA, SM-102, ALC-0315, C12-200, LP01 and 5A2-SC8. The helper lipids include any one or more of 1,2-dioleyl-sn-glyceryl-3-phosphatidylethanolamine (DOPE), 1,2-distearyl-sn-glyceryl-3-phosphatidylcholine (DSPC), dioleyl phosphatidylserine (DOPS), distearyl phosphatidylserine (DSPS), 1,2-distearyl-sn-glyceryl-3-phosphoethanolamine (DSPE), dipalmitoyl phosphatidylserine (DPPS), 1,2-dipalmitoyl-sn-glyceryl-3-phosphatidylcholine (DPPC), 1,2-dioleyl-sn-glyceryl-3-phosphatidylcholine (DOPC), dipalmitoyl phosphatidylglycerol (DPPG), oleoyl phosphatidylcholine (POPC), 1-palmitoyl-2-oleoyl phosphatidylethanolamine (POPE), 1,2-palmitoyl-sn-glyceryl-3-phosphoethanolamine (DPPE), 1,2-dimmyristoyl-sn-glyceryl-3-phosphoethanolamine (DMPE), distearoyl phosphatidylethanolamine (DSPE) and 1-stearoyl-2-oleoyl phosphatidylethanolamine (SOPE). The structural lipids include any one or more of cholesterol, nonsterosterol, sitosterol, ergosterol, campesterol, stigmasterol, campesterol, tomatidine, ursolic acid, α-tocopherol, coprosterol and corticosteroid. The polymer-modified lipids includes any one or more of 1,2-dimyristoyl-rac-glyceryl-3-methoxy polyethylene glycol 2000 (DMG-PEG2000), DMG-PEG2000-mannose, cholesterol-PEG2000, 1,2-dimyristoyl-sn-glycol methoxy-polyethylene glycol (PEG-DMG), dimyristoyl glycol-polyethylene glycol (PEG-c-DMG), polyethylene glycol-dimyristoyl glycol (PEG-C14), PEG-1,2-dimyristoyloxypropyl-3-amine (PEG-c-DMA), 1,2-distearoyl-sn-glyceryl-3-phosphoethanolamine-N-[amino (polyethylene glycol)] (PEG-DSPE), polyethylene glycol phosphatidylethanolamine (PEG-PE), PEG-modified ceramide, PEG-modified dialkyl amine, PEG-modified diacylglycerol, Tween-20, Tween-80, 1,2-dipalmitoyl-sn-glyceryl-methoxy polyethylene glycol (PEG-DPG), 4-O-(2′,3′-bis(tetradecanoyloxy) propyl-1-O-(ω)-methoxy (polyethoxy)ethyl) succinate (PEG-s-DMG), PEG-dialkoxy propyl (PEG-DAA), mPEG2000-1,2-di-O-alkyl-sn3-carbomoylglyceride (PEG-c-DOMG) and N-acetylgalactosamine ((R)-2,3-bis(octadecyloxy) propyl-1-(methoxy poly(ethylene glycol)2000) propyl carbamate)) (GalNAc-PEG-DSG).

In some embodiments, the N/P ratio of the liposome to the siRNA is in the range of 5-20, and preferably 6-10.

In some embodiments, a volume ratio of a solution of the siRNA to a solution of the liposome is (2-6):(1-2), and preferably 3:1.

In some embodiments, the encapsulation of the siRNA by the liposome is completed using a microfluidic encapsulation device.

In some embodiments, the siRNA is dissolved in DEPC water.

In another aspect, the present disclosure provides a method for controlling rice blast, where a MOHS1 gene is used as a target for controlling rice blast, and the MOHS1 gene includes a nucleotide sequence as shown in SEQ ID NO: 1 in Sequence Listing.

In some embodiments, the method refers to utilizing a siRNA targeting silence of the MOHS1 gene to achieve the purpose of controlling Magnaporthe oryzae.

In some embodiments, the siRNA includes a nucleotide sequence as shown in any one or more of SEQ ID NOs: 38-39, SEQ ID NOs: 40-41, SEQ ID NOs: 42-43, SEQ ID NOs: 44-45, SEQ ID NOs: 46-47, SEQ ID NOs: 48-49, SEQ ID NOs: 50-51, SEQ ID NOs: 52-53, SEQ ID NOs: 54-55, SEQ ID NOs: 16-17, SEQ ID NOs: 56-57, SEQ ID NOs: 58-59, SEQ ID NOs: 60-61, SEQ ID NOs: 62-63, SEQ ID NOs: 64-65, SEQ ID NOs: 66-67, and SEQ ID NOs: 68-69 in the Sequence Listing.

In some embodiments, the siRNA includes a nucleotide sequence as shown in any one or more of SEQ ID NOs: 48-49, SEQ ID NOs: 50-51, SEQ ID NOs: 16-17, SEQ ID NOs: 58-59, and SEQ ID NOs: 68-69 in the Sequence Listing.

In some embodiments, the siRNA includes a nucleotide sequence as shown in any one or more of SEQ ID NOs: 16-17 in the Sequence Listing.

In some embodiments, the method refers to spraying a solution containing the siRNA and/or a nucleic acid pesticide onto a plant.

To further explore the control effect of siMohs-10 on rice blast, the siMohs-10 and spores of the Magnaporthe oryzae are mixed and inoculated onto the leaves of Oryza sativa L. It has been found that the area of the lesion of Oryza sativa L. treated with the siMohs-10 is smaller than that of a control, indicating that the siRNA targeting the MOHS1 gene can reduce the pathogenicity of the Magnaporthe oryzae, and further proving that the siMohs-10 can be used for controlling rice blast.

In some embodiments, the plant is a Poaceae plant.

In some embodiments, the siRNA has a working time of 1-24 h.

The optimal working concentration range and working time of the siRNA are 6.25-25 μM and 8 h, respectively. The Magnaporthe oryzae is treated with different concentrations of siMohs-10 to explore the effects of different concentrations of siRNAs on the formation of an appressorium of the Magnaporthe oryzae. The results have shown that when the concentration of the siRNA is 25 μM, after the Magnaporthe oryzae is treated for 4 h, the inhibitory effect on the formation of the appressorium of the Magnaporthe oryzae is most obvious, and the inhibitory effect on the growth of the Magnaporthe oryzae can last for 8 h.

In a further aspect, the present disclosure provides a method for interfering development and/or pathogenicity of an appressorium of Magnaporthe oryzae, including utilizing a siRNA targeting silence of a MOHS1 gene, where the MOHS1 gene has a nucleotide sequence as shown in SEQ ID NO: 1 in Sequence Listing.

The beneficial effects of the present disclosure include:

1. in the present disclosure, the effects of multiple siRNA molecules targeting key genes for growth and development of the Magnaporthe oryzae on the formation of the appressorium of the Magnaporthe oryzae are investigated. After comparison, the siRNA targeting the silence of the MOHS1 gene has the best effect.

2. A target gene MOHS1 that plays a role in the control of the Magnaporthe oryzae, is provided, and the siRNA molecule designed according to this target gene has an optimal working concentration of 25 μM, and it effectively inhibits the formation of the appressorium of the Magnaporthe oryzae within 8 h. The encapsulation with the nanomaterial can prolong the acting time of the siRNA.

3. The siRNA molecule for resisting infection with the Magnaporthe oryzae provided by the present disclosure can be prepared into a nucleic acid pesticide, which is sprayed onto the leaves of Oryza sativa L. to avoid a tedious process of cultivating genetically modified crops and the concerns of consumer groups, thereby providing a novel method for controlling rice blast.

### BRIEF DESCRIPTION OF THE DRAWINGS

To describe the technical solutions in the embodiments of the present disclosure or in the prior art more clearly, the following briefly describes the accompanying drawings required for describing the embodiments or the prior art. Apparently, the accompanying drawings in the following description show some embodiments of the present disclosure, and a person of ordinary skill in the art may still derive other drawings from these accompanying drawings without creative efforts.

FIG. 1: absorption of siRNAs in vitro by Magnaporthe oryzae (fluorescence signal distribution diagram): with a scale bar=20 μm.

FIG. 2A: the appressorium formation rate of Magnaporthe oryzae treated with siRNAs targeting different genes. The data is analyzed by Student's t-test, ****p<0.0001; and DDW, siGFP-255 and siMcherry-362 are employed as negative controls.

FIG. 2B: The representative photographs of spore germination of the rice blast fungus, which are responded with the results shown in FIG. 2A. Scale bar=20 μm, DDW and siGFP-255 are used as negative controls.

FIG. 3: a distribution diagram of different siRNA positions on the target gene sequence.

FIG. 4: comparison of the inhibitory effects of different siRNA fragments on the Magnaporthe oryzae: with a scale bar=20 μm.

FIG. 5: a formation rate of an appressorium of Magnaporthe oryzae after treatment with different concentrations of siRNAs for different times.

FIG. 6A: the appressorium formation rate of Magnaporthe oryzae incubated with siMohs-10 encapsulated by nanomaterials for 4 h, 8 h. The data is analyzed by Student's t-test, ****p<0.0001; and DDW is employed as a negative control.

FIG. 6B: The representative photographs of spore germination of the rice blast fungus incubated with siMohs-10 encapsulated by nanomaterials for 8 h, which are responded with the results shown in FIG. 6A. Scale bar=20 μm. DDW is employed as a negative control.

FIG. 7: effects of siRNA spraying on the pathogenicity of Magnaporthe oryzae.

### DETAILED DESCRIPTION OF THE INVENTION

The present disclosure is further described in detail hereafter in conjunction with the accompanying drawings and specific embodiments. The embodiments are only used for explaining the present disclosure, and are not intended to limit the scope of the present disclosure. Unless otherwise specified, the experimental methods used in the following examples are all conventional methods; and unless otherwise specified, the materials, reagents and the like as used are reagents and materials that are commercially available.

In regard of the siRNA in the present disclosure, the nomenclature of it is as follows: si+gene name-number, and for example siMohs-10 means the 10th pair of siRNAs targeting silence of a MOHS1 gene; and also for example siGFP-255 refers to the siRNA targeting silence of a GFP gene. Although the Mohs in the accompanying drawings provided by the present disclosure and the MOHS1 in the specification are inconsistent in wording, they are named by the inventor according to his or her habit, and the two are essentially the same and have the same meaning.

### Example 1: The Magnaporthe oryzae had the Ability to Absorb siRNA Molecules from the External Environment

In this example, in order to determine whether Magnaporthe oryzae can uptake RNA molecules in the external environment, the spores of the Magnaporthe oryzae were treated with a siRNA carrying a fluorescent label (taking siGFP-255 as an example, see Sequence Listing for the specific sequence).

Preparation of the siRNA carrying a fluorescent label: GenePharma was entrusted to synthesize a segment of siRNA (taking siGFP-255 as an example), and a fluorescent label was added to the 5′ terminal of a sense strand. The siRNA carrying a fluorescent label was dissolved to a final concentration of 20 μM, and stored for later use. It should be noted that the siGFP-255 (SEQ ID NOs: 2-3) referred to a siRNA targeting silence of a GFP gene.

Collection of liquid of spores of Magnaporthe oryzae: a culture plate of Magnaporthe oryzae (Guy11) grown for 8-10 d was washed with sterile water, and the washing solution was filtered into a centrifuge tube and centrifuged at 7,000 rpm for 4 min, and the supernatant was discarded. The spore concentration was adjusted to 1×10⁵ spores/mL by a hemacytometer. The Guy11 was a natural strain isolated from a rice blast sample in the field of French Guiana region (Leung et al. 1988) “Leung H, Borromeo E S, Bernardo M A, Notteghem J L. 1988. Genetic analysis of virulence in the rice blast fungus Magnaporthe grisea. Phytopathology. 78(9):1227-1233. doi: 10.1094/Phyto-78-1227”. It was one of the model strains for research of the Magnaporthe oryzae used in many laboratories around the world. This strain is a highly virulent strain to quite a lot rice cultivars, could well reflect the actual pathogenicity and occurrence of the Magnaporthe oryzae, and was mentioned in hundreds of articles such as “Identification and Characterization of MPG1, a Gene Involved in Pathogenicity from the Rice Blast Fungus Magnaporthe grisea” [1].

Appressorium induction: a hydrophobic membrane was rinsed with sterile water, then wiped with 70% ethanol, and dried in the air, and then the dried hydrophobic membrane was placed in a moisturizing box. A siGFP carrying a fluorescently label was mixed with the spores of the Magnaporthe oryzae, then spotted onto the hydrophobic membrane, and incubated in vitro for 4 h, 8 h, and 12 h, respectively. After completion of the incubation, the surface liquid of the hydrophobic membrane was pipetted and observed under a fluorescence microscope.

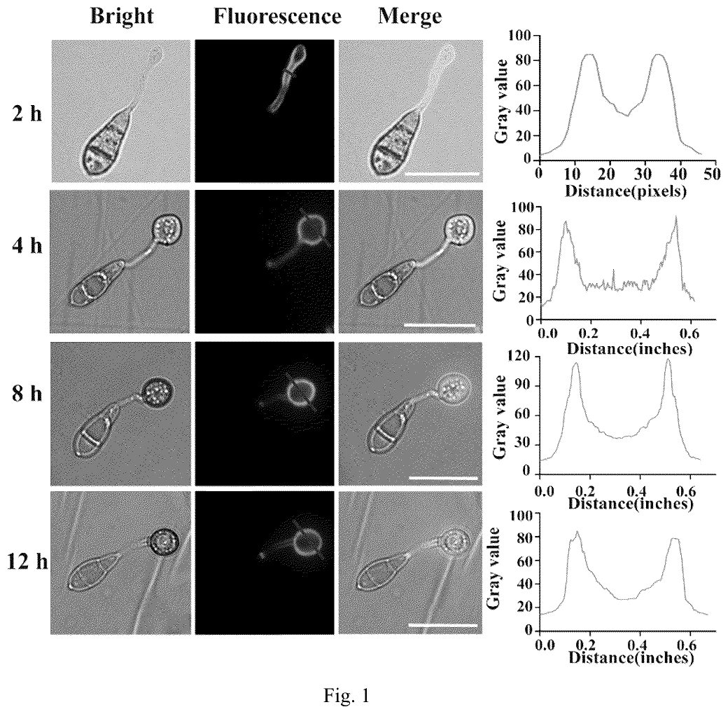

The results were shown in FIG. 1. After the siRNA carrying a fluorescent label was incubated with the spores in vitro for 2 h, fluorescent signals had been enriched on the germ tubes of the spores. The fluorescent signals in the germ tubes could reach 40%. From 4-8 h, the fluorescent signals in the appressorium continued to increase, but at 8-12 h, the fluorescent signals began to decrease, indicating that the siRNA had a certain shelf life. The results showed that the spores of the Magnaporthe oryzae could uptake the siRNA from the moment they germinated the germ tubes. In addition, the fluorescence signals are mainly concentrated on the surfaces of the germ tubes and early appressoria, with a small amount of fluorescence enriched inside the appressoria, but no fluorescence enriched in the spores.

### Example 2: Screening of siRNAs Targeting Different Key Genes of Magnaporthe oryzae

Design and synthesis of siRNAs: combined with the results of the aforementioned examples, this example used key genes that regulate the growth and development of the Magnaporthe oryzae as target genes, and conducted designing of siRNAs according to the methods for designing siRNAs on the website (http://siDirect2.rnai.jp), and combined with bioinformatics and gene data analysis, the possibility of targeting the genome of Oryza sativa L. and other genes of the Magnaporthe oryzae was reduced, so as to obtain siRNAs (siChs1-1 (SEQ ID NOs: 6-7), siChs1-3 (SEQ ID NOs: 8-9), siChs3-1 (SEQ ID NOs: 10-11), siChs3-2 (SEQ ID NOs: 12-13), siChs7-1959 (SEQ ID NOs: 14-15), siMohs-10 (SEQ ID NOs: 16-17), siBuf1-156 (SEQ ID NOs: 18-19), siBuf1-624 (SEQ ID NOs: 20-21), siMGG_04732-488 (SEQ ID NOs: 22-23), siMGG_04732-1172 (SEQ ID NOs: 24-25), siMGG_04876-381 (SEQ ID NOs: 26-27), siMGG_04876-480 (SEQ ID NOs: 28-29), siHtfg-1318 (SEQ ID NOs: 30-31), siRgs4-989 (SEQ ID NOs: 32-33), siRgs7-795 (SEQ ID NOs: 34-35), and siRgs7-1595 (SEQ ID NOs: 36-37), see the Sequence Listing for the specific sequences). The double-stranded molecules of the siRNAs of the present disclosure were directly synthesized as entrusted to a biological company (Generay, Shanghai), and the synthesized product was dissolved to a final concentration of 10 μM by addition of water, and sub-packaged for storage.

The collection of the spores of the Magnaporthe oryzae was conducted with reference to that in Example 1.

Co-incubation of siRNA and Magnaporthe oryzae: the hydrophobic membrane for inducing appressorium formation was cleaned in advance, and then wiped with 70% ethanol, and the wiped hydrophobic membrane was placed in a moisturizing box. The siRNA was mixed with the liquid of spores of the Magnaporthe oryzae and then spotted onto the center of the treated hydrophobic membrane. At this time the final concentration of the siRNA was 5 μM. After incubation for 4 h, the formation of appressoria was observed and recorded.

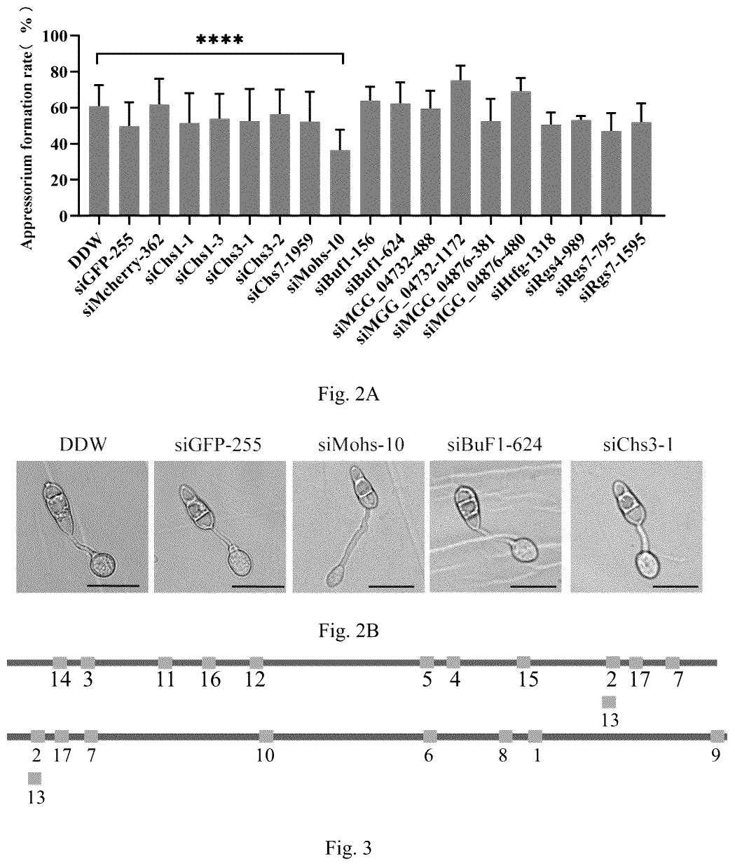

The results were shown in FIG. 2A and FIG. 2B. Compared with negative controls (DDW. siGFP-255 and siMcherry-362 (SEQ ID NOs: 4-5) and siRNAs targeting other genes, the siRNA (siMohs-10, SEQ ID NOs: 16-17) targeting the MOHS1 gene of the Magnaporthe oryzae could significantly reduce the formation proportion of the appressoria of the Magnaporthe oryzae, and the formation rate of the appressoria of the Magnaporthe oryzae was decreased by about 25%; and at the same time, the morphology of the spores of the Magnaporthe oryzae was observed 4 h after treatment. Compared with the control, the Magnaporthe oryzae treated with siMohs-10 had elongated germ tubes, smaller morphology of appressoria, and lighter melanin layers of the appressoria. The results of this example showed that exogenously added siMohs-10 targeting the MOHS1 gene of the Magnaporthe oryzae could inhibit the morphological construction of the appressorium of the Magnaporthe oryzae.

### Example 3: Design of siRNA Targeting MOHS1 Gene

According to the results of the aforementioned Example 2, it was indicated that the MOHS1 gene was an effective target gene for controlling the Magnaporthe oryzae. Therefore, in order to select the siRNA with the best effect in targeting silence of the MOHS1 gene, this example conducted designing of siRNAs for the target silencing regions present in the full length of the MOHS1 gene (the specific sequences were shown in the Sequence Listing, and the positions were shown in FIG. 3), and a total of 17 pairs of siRNAs (including the siRNA siMohs-10 involved in Example 2) were obtained. The design principles of siRNAs were described in Example 2.

### Example 4: Comparison of the Inhibitory Effects of Different siRNAs Targeting MOHS1 Gene on Magnaporthe oryzae

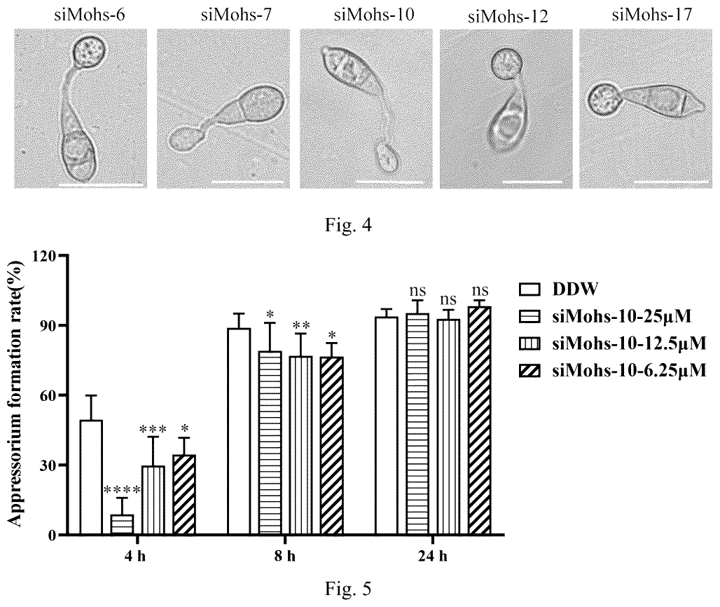

4 siRNAs (siMohs-6, siMohs-7, siMohs-12 and siMohs-17) designed in Example 3 were randomly selected for comparison of their antibacterial effects with those of the siRNA siMohs-10 in Example 2. By incubating siRNAs at the same concentration with the Magnaporthe oryzae, the effects on the Magnaporthe oryzae were observed and recorded at 4 h. The results were shown in FIG. 4. At 4 h, the Magnaporthe oryzae applied with the siMohs-7 and siMohs-10 molecules had hindered appressorium formation and smaller appressorium morphology, compared with other siRNAs; however, the inhibitory effect of the siMohs-10 was better than that of the siMohs-7. The results of this example showed that the siMohs-10 was the siRNA molecule with the best effect in targeting the MOHS1 gene.

### Example 5: Effects of Different Concentrations of siRNAs on Formation of Appressoria of Magnaporthe oryzae

Combined with the results of Example 3 and Example 4, this example selected siMohs-6, siMohs-7, siMohs-10, siMohs-12 and siMohs-17 as research objects to explore the effects of different concentrations of siRNAs on the formation of the appressorium of Magnaporthe oryzae. The dissolution and storage manners of siRNA molecules were the same as those in Example 1.

In this example, the siRNA was first prepared into mother liquors with initial concentrations of 50 μM, 25 μM, and 12.5 μM, and then mixed with the liquid of spores of the Magnaporthe oryzae at the adjusted concentration in Example 3 to form siRNAs with final concentrations of 25 μM, 12.5 μM, and 6.25 μM, which were then spotted onto the center of the hydrophobic membrane and incubated for 4 h, 8 h, and 24 h, respectively, to observe and record the formation of appressoria. The results were shown in FIG. 5. The appressorium formation rate of the Magnaporthe oryzae co-incubated with the siMohs-10 for 4 h was significantly lower than that of the control, and with the increase of the concentration of the siMohs-10, the appressorium formation rate was lower. That was, treatment with 25 μM of the siMohs-10 had the best effect. After 8 h co-incubation of the Magnaporthe oryzae with the siMohs-10, compared with the control, the siRNA could still effectively inhibit the formation of the appressoria of the Magnaporthe oryzae, but the inhibitory effects of different concentrations of siRNAs were relatively consistent. By 24 h, the appressoria of the Magnaporthe oryzae gradually formed, and the inhibitory effect of siMohs-10 gradually weakened. The results of this example showed that the inhibitory effect of the treatment with 25 μM of the siMohs-10 for 4 h on the formation of the appressoria of the Magnaporthe oryzae was the most obvious; and the inhibitory effect of the siMohs-10 on the growth of the Magnaporthe oryzae could last for 8 h. The aforementioned experiments were also conducted on siMohs-6, siMohs-7, siMohs-12 and siMohs-17, but the effects were all not as good as that of siMohs-10.

### Example 6: Nanomaterials could Prolong the Duration of the Inhibitory Effect of siRNA on Magnaporthe oryzae

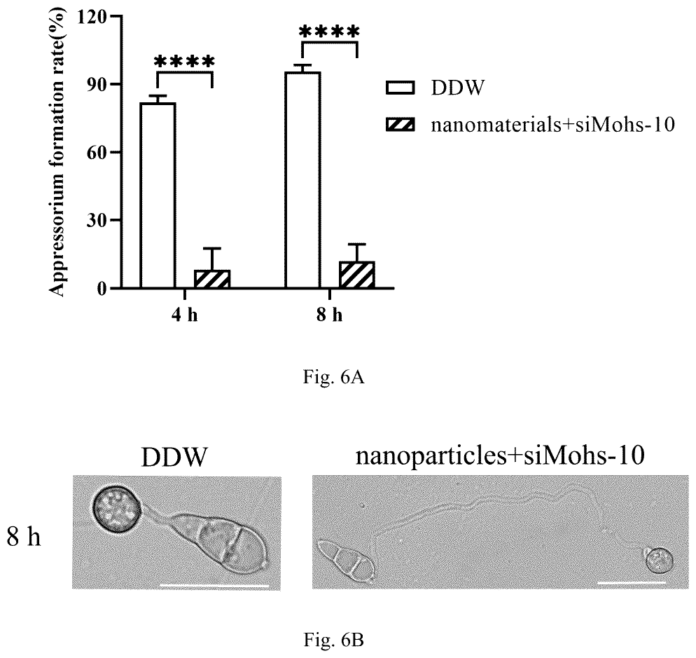

Combined with the aforementioned results of Example 5, this example utilized a nanomaterial (this example preferably used the lipid encapsulation reagent Lipid mixer for RNA/DNA delivery provided by JIKE Biopharmaceutical Technology (Hangzhou) Co., Ltd., Cat. No. P201-6 mM) to encapsulate and thus prolong the inhibitory effects of the 5 siMohs molecules on the growth of the Magnaporthe oryzae. First, the synthesized siRNA powder was dissolved in DEPC-treated water (purchased from Vazyme), and at this time a final concentration of the solution was 10 μM. Using the nanoencapsulation microfluidic instrument and chip provided by Shanghai Zheben Instrument Technology Co., Ltd., the siRNA solution (aqueous phase) and the nanomaterial (organic phase) were introduced into the instrument from both an aqueous phase position and an organic phase position with a disposable micro-medical syringe in a volume ratio of 3:1, and the resulting mixture was diluted to 1 mL with DEPC-treated water for storage. Because the liposome contains ethanol, in order to avoid the influence of ethanol on this experiment, it is necessary to remove ethanol using an ultrafiltration tube (Amicon Ultra centrifugal ultrafiltration tube, 50 kDaMWCO). At this time, the concentration of the siRNA was 3 μM. The siRNA encapsulated by the nanomaterial was co-incubated with the spores of the Magnaporthe oryzae for 4 h and 8 h, and the formation of appressoria was observed and recorded. The results were shown in FIG. 6A and FIG. 6B. Compared with the control (DDW), the formation of the appressoria was significantly inhibited after the Magnaporthe oryzae was treated with the siMohs-10 encapsulated by the nanomaterial for 4 h and 8 h. After 8 h of treatment, the Magnaporthe oryzae had elongated germ tubes, relatively smaller appressorium morphology, and no normal formation of the melanin layer. Furthermore, compared with the results of Example 5. although the concentration of the siMohs-10 molecular solution encapsulated by the nanomaterial (liposome+siMohs-10 group) was only 3 μM, while the concentration of the siRNA in Example 3 was 6.25-25 μM, the former has a significantly better inhibitory effect on the Magnaporthe oryzae than the latter. Specifically, it was manifested as whether co-incubation for 4 h or 8 h, the appressorium formation rate of the nanomaterial+siMohs-10 group was less than 15% (FIG. 6A); on the contrary, despite the increase in the concentration of the siRNA, the inhibitory effect of the siMohs-10 molecular solution alone would be significantly decreased over time. That was, when co-incubated for 4 h, the Magnaporthe oryzae had an appressorium formation rate of 10-30%, and at 8 h, the appressorium formation rate was increased to about 80%, and until after 24 h, the siMohs-10 molecules could no longer interfere with the growth of the Magnaporthe oryzae (FIG. 5), which might be because the siRNA was easily decomposed and could not be exposed to the external environment for a long time. Further, in terms of the morphology of the Magnaporthe oryzae, compared with treatment with the siMohs-10 molecule alone (FIG. 4), the germ tubes germinated from the spores of the Magnaporthe oryzae treated with the nanomaterial+siMohs-10 group were more slender, indicating that this treatment condition could better interfere with the growth process of the Magnaporthe oryzae. In view of the above, the nanomaterial could not only prolong the acting time of siMohs-10, but also improve the acting effect of siMohs-10. The aforementioned experiments were also conducted on siMohs-6, siMohs-7, siMohs-12 and siMohs-17, and similar conclusions were also obtained.

In view of the fact that the liposome could enhance the acting effect of the siMohs molecule, this example used the siMohs-10 as the research object to explore the synergistic effects of different liposome components. The experimental steps were the same as described above. The specific results were shown in Table 1.

TABLE 1
Effects of liposome components on acting effect of siMohs-10

| | | | Appressorium |
|---|---|---|---|
| | | | formation rate |
| Liposome | | Acting time | after 72 h of co- |
| Name | Liposome source | of siMohs-10 | incubation (%) |
| Lipid mixer for | JIKE | <72 h | 25% |
| RNA/DNA | Biopharmaceutical | | |
| delivery | Technology | | |
| | (Hangzhou) Co., Ltd. | | |
| DOPC + | Xi'an Ruixi | <48 h | 40% |
| cholesterol | Biotechnology Co., | | |
| LNP | Ltd. (R-PC-01) | | |

As shown in Table 1, the first liposome (Lipid mixer for RNA/DNA delivery) worked best when used in combination with the siMohs-10. It could prolong the acting time of the siMohs-10 and synergistically improve the inhibitory effect of the siMohs-10 on the Magnaporthe oryzae optimally. This might be because the liposome had a better RNA encapsulation effect and can better protect the siRNA from degradation, thereby helping the siMohs-10 to play its role to the maximum extent.

### Example 7: Effect of siRNA Spray on Controlling Rice Blast

In this example, the acting effect of the siMohs on the Magnaporthe oryzae was further explored by spray inoculation.

Planting of Oryza sativa L.: the Oryza sativa L. was subjected to treatment of seed soaking and germination hastening, Oryza sativa L. (CO-39) seeds with a consistent growth status were selected. 22-25 seeds were sowed in each pot, and when the Oryza sativa L. grew to a three-leaf-one stage, the Oryza sativa L. plant was covered with a fresh-keeping bag to keep moisture and prevent liquid from overflowing during spraying.

Collection and acquisition of spore liquid of Magnaporthe oryzae: a plate of the Magnaporthe oryzae (Guy11) grown for 10 d was selected, the hyphae were scraped from the plate with a disposable applicator bar using sterile water, the resulting liquid was filtered, and the filtrate was the spore liquid. Then the concentration of the spore liquid was adjusted to 2×10⁵ spores/mL using a hemacytometer, and then diluted to 1×10⁵ spores/mL using a 0.3% gelatin solution.

Experiment of pathogenicity of Oryza sativa L.: in this example, siRNA molecules were selected and mixed with spores of the Magnaporthe oryzae for spray inoculation. The siRNA was mixed with the spore liquid of the Magnaporthe oryzae in equal volumes. The final concentration of the siRNA was 5 μM, and 2 mL of the mixed solution of the siRNA and the spores was sprayed onto the Oryza sativa L. in each pot. Then the plants were placed into an incubator and cultured at 22° C. under dark conditions for 2 d, and then irritated with light at 25° C. with light: dark=10 h: 12 h. The incidence of the leaves of the Oryza sativa L. was observed, and the leaves were cut to count the incidence results.

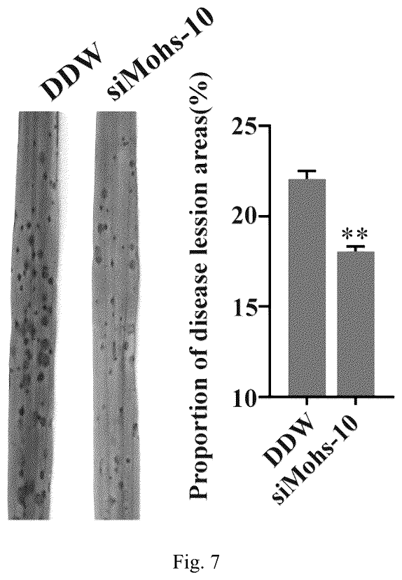

The results were shown in FIG. 7. Compared with the control, the size of the lesions on the leaves of the Oryza sativa L. was significantly reduced after treatment with the siMohs-10, indicating that the pathogenicity of the spores of the Magnaporthe oryzae on the leaves of the Oryza sativa L. was significantly reduced. The aforementioned experiment was also performed on siMohs-6, siMohs-7, siMohs-12 and siMohs-17, but the siMohs-10 had the best antibacterial effect. The results of this example showed that the siMohs-10 targeting the key gene MOHS1 of the Magnaporthe oryzae could control the Magnaporthe oryzae. The results provided a certain theoretical and experimental basis for SIGS to control the Magnaporthe oryzae.

The embodiments described above provide a detailed description of the technical solutions of the present disclosure. It should be understood that the above are only specific embodiments of the present disclosure and are not intended to limit the present disclosure. Any modifications, supplements or similar substitutions made within the scope of the principles of the present disclosure should be included in the claimed scope of the present disclosure.

### REFERENCES

[1] Talbot N J, Ebbole D J, Hamer J E. Identification and characterization of MPG1, a gene involved in pathogenicity from the rice blast fungus Magnaporthe grisea. Plant Cell. 1993 November; 5(11):1575-90.

1. A nucleic acid pesticide for controlling Magnaporthe oryzae, comprising a siRNA targeting silence of a MOHS1 gene of Magnaporthe oryzae, wherein the MOHS1 gene comprises a nucleotide sequence as shown in SEQ ID NO: 1 in Sequence Listing.

2. The nucleic acid pesticide according to claim 1, wherein the siRNA comprises a nucleotide sequence as shown in any one or more of SEQ ID NOs: 38-39, SEQ ID NOs: 40-41, SEQ ID NOs: 42-43, SEQ ID NOs: 44-45, SEQ ID NOs: 46-47, SEQ ID NOs: 48-49, SEQ ID NOs: 50-51, SEQ ID NOs: 52-53, SEQ ID NOs: 54-55, SEQ ID NOs: 16-17, SEQ ID NOs: 56-57, SEQ ID NOs: 58-59, SEQ ID NOs: 60-61, SEQ ID NOs: 62-63, SEQ ID NOs: 64-65, SEQ ID NOs: 66-67, and SEQ ID NOs: 68-69 in the Sequence Listing.

3. The nucleic acid pesticide according to claim 1, wherein the siRNA comprises a nucleotide sequence as shown in any one or more of SEQ ID NOs: 48-49, SEQ ID NOs: 50-51, SEQ ID NOs: 16-17, SEQ ID NOs: 58-59, and SEQ ID NOs: 68-69 in the Sequence Listing.

4. The nucleic acid pesticide according to claim 1, wherein the siRNA comprises a nucleotide sequence as shown in any one or more of SEQ ID NOs: 16-17 in the Sequence Listing.

5. The nucleic acid pesticide according to claim 1, wherein the siRNA has a working concentration range of 1-100 μM.

6. The nucleic acid pesticide according to claim 1, wherein the siRNA is encapsulated by a nanomaterial.

7. The nucleic acid pesticide according to claim 6, wherein the nanomaterial is a liposome.

8. The nucleic acid pesticide according to claim 7, wherein a volume ratio of a solution of the siRNA to a solution of the liposome is (2-6):(1-2).

9. The nucleic acid pesticide according to claim 7, wherein a volume ratio of the solution of the siRNA to the solution of the liposome is 3:1.

10. The nucleic acid pesticide according to claim 7, wherein the encapsulation of the siRNA by the liposome is completed using a microfluidic encapsulation device.

11. The nucleic acid pesticide according to claim 7, wherein the siRNA is dissolved in DEPC water.

12. A method for controlling rice blast, wherein a MOHS1 gene acts as a target for controlling rice blast, and the MOHS1 gene comprises a nucleotide sequence as shown in SEQ ID NO: 1 in Sequence Listing.

13. The method according to claim 12, wherein the method refers to utilizing a siRNA targeting silence of the MOHS1 gene to achieve the purpose of controlling rice blast.

14. The method according to claim 13, wherein the siRNA comprises a nucleotide sequence as shown in any one or more of SEQ ID NOs: 38-39, SEQ ID NOs: 40-41, SEQ ID NOs: 42-43, SEQ ID NOs: 44-45, SEQ ID NOs: 46-47, SEQ ID NOs: 48-49, SEQ ID NOs: 50-51, SEQ ID NOs: 52-53, SEQ ID NOs: 54-55, SEQ ID NOs: 16-17, SEQ ID NOs: 56-57, SEQ ID NOS: 58-59, SEQ ID NOs: 60-61, SEQ ID NOs: 62-63, SEQ ID NOs: 64-65, SEQ ID NOs: 66-67, and SEQ ID NOs: 68-69 in the Sequence Listing.

15. The method according to claim 13, wherein the siRNA comprises a nucleotide sequence as shown in any one or more of SEQ ID NOs: 48-49, SEQ ID NOs: 50-51, SEQ ID NOs: 16-17, SEQ ID NOs: 58-59, and SEQ ID NOs: 68-69 in the Sequence Listing.

16. The method according to claim 13, wherein the siRNA comprises a nucleotide sequence as shown in any one or more of SEQ ID NOs: 16-17 in the Sequence Listing.

17. The method according to claim 13, comprising spraying a solution containing the siRNA and/or a nucleic acid pesticide onto a plant, wherein and the nucleic acid pesticide comprises a siRNA targeting silence of a MOHS1 gene of Magnaporthe oryzae, wherein the MOHS1 gene comprises a nucleotide sequence as shown in SEO ID NO: 1 in Sequence Listing.

18. The method according to claim 17, wherein the plant is a Poaceae plant.

19. The method according to claim 13, wherein the siRNA has a working time of 1-24 h.

20. A method for interfering development and/or pathogenicity of an appressorium of Magnaporthe oryzae, comprising utilizing a siRNA targeting silence of a MOHS1 gene, wherein the MOHS1 gene has a nucleotide sequence as shown in SEQ ID NO: 1 in Sequence Listing.
